(19) United States
(12) Patent Application Publication
(10) Pub. No.: US 2009/0028802 A1
(43) Pub. Date: Jan. 29, 2009

(54) ORGANOLEPTICALLY ACCEPTABLE INDOLE SEROTONIN RECEPTOR AGONIST ORAL DOSAGE FORMULATIONS AND METHODS OF USING THE SAME

(76) Inventor: Toru Hibi, San Jose, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/174,467

(22) Filed: Jul. 16, 2008

Related U.S. Application Data
(60) Provisional application No. 60/961,737, filed on Jul. 23, 2007.

(51) Int. Cl. A61K 9/68; A61K 31/403; A61K 31/421; A61K 31/4196; A61P 25/06; A61K 31/454; A61K 31/4045

(52) U.S. Cl. 424/48; 514/411; 514/376; 514/414; 514/383; 514/323; 514/415

(57) ABSTRACT

Organoleptically acceptable oral dosage formulations of an indole receptor serotonin agonist, and methods of making and using the same, are provided. An aspect of the formulations is that they include an indole receptor serotonin agonist and a masking component. In certain embodiments, the masking component includes one or more of an amino acid and an organic acid. The subject invention finds use in a variety of applications.

### CROSS-REFERENCE TO RELATED APPLICATIONS

Pursuant to 35 U.S.C. §119 (e), this application claims priority to the filing date of U.S. Provisional Patent Application Ser. No. 60/961,737 filed Jul. 23, 2007; the disclosure of which is herein incorporated by reference.

### INTRODUCTION

Although the epidemiology of headache disorders is only partly documented, taken together, headache disorders are extraordinarily common. It has been estimated that worldwide approximately 240 million people have migraine attacks each year. The National Headache Foundation states that more than 29.5 million Americans suffer from migraine headaches, with women being affected three times more often than men. In addition, in developed countries, tension type or “stress” headaches are estimated to affect two-thirds of all adult males and over 80% of adult females. Less well known is the prevalence of chronic daily headaches although the World Health Organization (WHO) estimates that one adult in 20 has a headache every or nearly every day. Trigeminal neuralgia is not a common disorder but the pain associated with trigeminal neuralgia attacks has been described as among the most severe known to mankind.

Not only are headaches painful, but headache disorders can be disabling to afflicted individuals. Worldwide, according to the WHO, when analyzing all causes for “years lived with disability” migraine headaches were rated 19th on the list. Headache disorders may impose substantial hardships and burdens on the afflicted individuals including personal suffering, impaired quality of life and high financial cost. Repeated headache attacks, and often the constant fear of the next one, can damage an individual's family life, social life and their productivity at their place of employment. For example, it is estimated that social activity and work capacity are reduced in almost all migraine sufferers and in 60% of tension headache sufferers. Finally, the long-term effort of coping with a chronic headache disorder may also predispose an individual to other illnesses. For example, depression is three times more common in people with migraine or severe headaches than in healthy individuals.

Triptan-type drugs, which are modified forms of serotonin (5-hydroxytryptamine; 5-HT), have been developed for the treatment of migraine headaches. Triptan-type drugs are serotoninergic agents that exhibit receptor-selective properties. Although the principal mechanism of action of triptan-type drugs is still under research, it is understood that they relieve the various symptoms of a migraine headache by inhibiting the over activity of trigeminal nerve terminals through serotonin 5-HT1B, 5-HT1D, 5-HT1F receptors that exist in blood vessels in the brain and trigeminal nerves; and by inhibiting inflammation around blood vessels, hyperlucency and vasodilation.

Various formulations, such as injection formulations, oral formulations (e.g., tablets), and nasal formulations (e.g., nasal drops), have been developed for administration of triptan-type drugs. Nevertheless, there is continued interest in development of new delivery systems for triptan type drugs.

### SUMMARY

Organoleptically acceptable oral dosage formulations of an indole receptor serotonin agonist, and methods of making and using the same, are provided. An aspect of the formulations is that they include an indole receptor serotonin agonist and a masking component. In certain embodiments, the masking component includes one or more of an amino acid and an organic acid. The subject invention finds use in a variety of applications.

### DEFINITIONS

As used herein, the term “headache” includes migraine headache, cluster headaches, rebound headaches, and status migrainosus. “Migraine headache” refers to a subset of headaches characterized by unusually severe, unilateral, throbbing, headache pain, usually persisting for 4 hours to 72 hours, and often including one or more of the following symptoms: nausea, vomiting, sensitivity to light or sound. As used herein, “migraine” includes migraine headache, migraine without aura, migraine with aura, and migraine with aura but without headache. “Relapse headache” variously and interchangeably termed a “rebound,” “relapse,” “recurrent,” “follow on,” or “secondary” headache refers to headaches experienced by migraine patients after having experienced initial relief. A relapse headache may occur from 1 hour to 24 hours following initial relief from a migraine headache. Status migrainosus refers to a condition in which a patient, often with a previous history of migraine, suffers a continuous migraine. In status migrainosus, the pain is typical, unilateral and throbbing, and the patient is often disabled.

As used herein, “pain” includes acute pain, chronic pain and episodic pain.

As used herein, unless otherwise specified, the term “treatment” or “treating pain” refers to administration to an individual of an agent of interest wherein the agent alleviates or prevents a pathology for which the individual is being treated. “Treatment for headache pain”, “treatment of headache” or “treatment of head pain” refers to the alleviation or prevention of pain associated with headache disorders and trigeminal neuralgia.

As used herein, unless otherwise specified, the term “prevention”, “prophylaxis” or “preventing pain” refers to administration to an individual of an agent of interest wherein the agent alleviates or prevents a pathology for which the individual is being treated. “Prevention of headache pain”, “prevention of headache” or “prevention of head pain” refers to the alleviation or prevention of pain associated with headache disorders and trigeminal neuralgia.

As used herein, the term “indole serotonin receptor agonist” is used interchangeably with “triptan-type drug” and refers to an agent that has affinity for one or more of a 5-HT1B receptor, a 5-HT1D receptor, and a 5-HT1F receptor; and effects vasoconstriction of cerebral blood vessels and/or inhibition of pro-inflammatory neuropeptide release. An indole serotonin receptor agonist comprises a indole-3-alkylamine structure, as described in more detail below.

As used herein, the term “pharmaceutically acceptable salts” is used to describe those salts in which the anion (or cation) does not contribute significantly to the toxicity or pharmacological activity of the salt, and, as such, they are the pharmacological equivalents of the bases of the compounds to which they refer. Examples of pharmaceutically acceptable acids that are useful for the purposes of salt formation include but are not limited to hydrochloric, hydrobromic, hydroiodic, citric, acetic, benzoic, mandelic, fumaric, succinic, phosphoric, nitric, maleic, mucic, isethionic, palmitic, tannic and others. The active salt combinations of the pharmacologic ingredients may be the free acids, bases or as salts having anionic functional groups such as bitartrate, maleate, citrate, chloride, bromide, acetate and sulfate. The source of the functional groups may be natural or synthetic.

As used herein, “pharmaceutically acceptable carrier” or “suitable carrier” refers to a carrier that is conventionally used in the art to facilitate the storage, administration, and/or the healing effect of the agent.

As used herein, “therapeutically effective dose”, “therapeutically effective amount” or “an effective amount” refers to an amount of an analgesic agent that is useful for treating pain.

As used herein, “prophylactically effective dose”, “prophylactically effective amount” or “an effective amount” refers to an amount of an analgesic agent that is useful for preventing pain.

### DETAILED DESCRIPTION

Organoleptically acceptable oral dosage formulations of an indole receptor serotonin agonist, and methods of making and using the same, are provided. An aspect of the formulations is that they include an indole receptor serotonin agonist and a masking component. In certain embodiments, the masking component includes one or more of an amino acid and an organic acid. The subject invention finds use in a variety of applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As reviewed above, the present invention provides organoleptically acceptable indole receptor serotonin agonist oral dosage formulations, as well as methods for making and using the same. In further describing aspects of the invention in greater detail, embodiments of the organoleptically acceptable formulations are reviewed first in greater detail, followed by a review of certain protocols for making the formulations and a review of embodiments of applications in which the formulations find use.

### Organoleptically Acceptable Indole Receptor Serotonin Agonist Oral Dosage Formulations

As summarized above, aspects of the invention include organoleptically acceptable indole receptor serotonin agonist oral dosage formulations. As the formulations are organoleptically acceptable, they can contact the taste receptors of a recipient's mouth and be considered generally acceptable to the senses of the recipient, particularly to the sense of taste. The organoleptically acceptable formulations of this invention are oral formulations in which the unpleasant and bitter taste of an indole receptor serotonin agonist is sufficiently masked. When using the evaluation protocol reported in the Experimental Section below, the unpleasant and bitter taste of the indole receptor serotonin agonist is considered to be sufficiently masked if the composition scores a 1 or less, e.g., 0 or less, such as −1 or less, including −2.

### Indole Serotonin Receptor Agonists

As defined above, the term “indole serotonin receptor agonist” is used interchangeably with “triptan-type drug” and refers to an agent that has affinity for one or more of a 5-HT1B receptor, a 5-HT1D receptor, and a 5-HT1F receptor; and effects vasoconstriction of cerebral blood vessels and/or inhibition of pro-inflammatory neuropeptide release. Indole serotonin receptor agonists of interest include, but are not limited to, those compounds that are of Formula I:

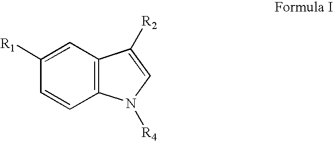

wherein R₁ is

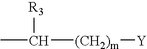

wherein Y is

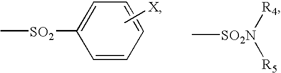

or a 5- or 6-membered cycloalkyl, wherein in some embodiments 1, 2, or 3 CH₂ groups are replaced by O, S, or NH, which cycloalkyl will in some embodiments by substituted with an oxo group;

X is H, C1-3-alkyl, C1-3-alkoxy, halogen, CF3, NO2 or NH2;

R₃ is H or C1-3-alkyl;

R₄ is H, C1-6-alkyl or C3-6-alkenyl;

R₅ is H, C1-3-alkyl, C3-6-alkenyl, aryl, aryl (C1-4alkylene or C5-7-cycloalkyl;

wherein R₂ is

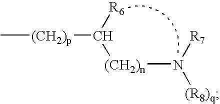

R₆ is H or (CH₂)ᵣ;

R₇ and R₈ are the same or different, and are each independently H, or C1-3-alkyl;

R₉ is H, C1-6-alkyl, or C3-6-alkenyl;

m, n, and r may be the same or different and are each independently an integer from 0 to 3, e.g., are each independently 0, 1, 2, or 3;

p is an integer that is 0 or 1; and

q is an integer that is 0 or 1;

with the proviso that when R₆ is (CH₂)ᵣ) and r is not zero, this group can be bound to the nitrogen atom of the radical NR₇(R₈)_{q} by a single bond, in which case q is zero. In some embodiments, the indole serotonin receptor agonist is a physiologically acceptable salt of a compound of Formula I, or a solvate of a compound of Formula I, or a pro-drug of a compound of Formula I. In some embodiments, e.g., the agonist is a succinate salt of a compound of Formula I.

In some embodiments, the indole serotonin receptor agonist is a compound of Formula I, where R₁ is CH₃HNSO₂CH₂; R₂ is —CH₂CH₂N(CH₃)₂; and R₄ is H. This compound is referred to as Sumatriptan.

In some embodiments, the indole serotonin receptor agonist is a compound of Formula I, where R₁ is

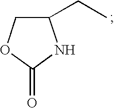

R₂ is —CH₂CH₂N(CH₃)₂; and R₄ is H. This compound is referred to as Zolmitriptan.

In some embodiments, the indole serotonin receptor agonist is a compound of Formula I, where R₁ is

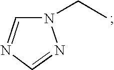

R₂ is —CH₂CH₂N(CH₃)₂; and R₄ is H. This compound is referred to as Rizatriptan.

In some embodiments, the indole serotonin receptor agonist is a compound of Formula I, where R₁ is CH₃HNSO₂CH₂; R₂ is

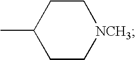

and R₄ is H. This compound is referred to as Naratriptan.

In some embodiments, the indole serotonin receptor agonist is a compound of Formula I, where R₁ is

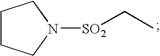

R₂ is —CH₂CH₂N(CH₃)₂; and R₄ is H. This compound is referred to as Almotriptan. In some embodiments, the indole serotonin receptor agonist is (R)-3-[(1 -methyl-2-pyrrolidinyl)methyl]-1H-indole-5-[2-(phenylsulfonyl)ethyl], also referred to as Eletriptan.

In some embodiments, the indole serotonin receptor agonist is R-(+) 3-methylamino-6-carboxamido-1,2,3,4-tetrahydrocarbazole, also referred to as Frovatriptan.

The agonist may be a free base or salt thereof. In some embodiments, e.g., the agonist is a succinate salt of the agonist, e.g., sumatriptan succinate.

The amount of indole serotonin receptor agonist present in the subject formulations may vary, so long as it is effective to acheive the intended purpose of the formulation, e.g., to provide pain relief to a subject in need thereof, as further reviewed below.

In addition to the indole serotonin receptor agonist active agent, the subject formulations also include a masking component. By masking component is meant a component that is made up of one or more agents which provides for sufficient masking of the indole serotonin receptor agonist bitterness to make the formulation organoleptically acceptable.

In certain embodiments, the masking component includes an amino acid masking agent and/or an organic acid masking agent. As such, the masking agent present may be one or more amino acids, one or more organic acids, or a combination of one or more amino acids and one or more organic acids.

Amino acids of interest include, but are not limited to: glycine, alanine, valine, leucine, isoleucine, serine, threonine, cysteine, cystine, methionine, aspartic acid, asparagine, glutamic acid, glutamine, arginine, lysine, 5-hydroxylysine, histidine, phenylalanine, tyrosine, tryptophan, 3-hydroxyproline, 4-hydroxyproline, proline, homocysteine, homocystine, homoserine, ornithine, citrulline, creatine, asparaginic acid, 3-aminopropanoic acid, theanine, 2-aminobutanoic acid, 4-aminobutanoic acid, 2-amino-2-methylpropanoic acid, 2-methyl-3-aminopropanoic acid, 2,6-diaminopimelic acid, 2-amino-3-phenylbutanoic acid, phenylglycine, canavanine, canaline, 4-hydroxyarginine, 4-hydroxyornithine, homoarginine, 4-hydroxyhomoarginine, β-lysine, 2,4-diaminobutanoic acid, 2,3-diaminopropanoic acid, 2-methylserine, 3-phenylserine betaine, sulfur-containing amino acids, such as taurine, cysteinesulfinic acid, methionine sulfoxide and methionine sulfone. In certain embodiments the amino acid masking agent is glutamic acid or glycine.

Organic acids of interest include, but are not limited to: glycolic acid, lactic acid, methyl lactic acid, palycarobxlyic acids, e.g., malic acid, citric acid, tartronic acid, tartaric acid, succinic acid, ascorbic acid, etc. In certain embodiments, the organic acid is chosen from citric acid, malic acid and ascorbic acid.

The amount of the masking agent that is present in the formulation is an amount sufficient (e.g., by itself or in combination with other masking agents of the masking component) to mask or hide the bitterness of the indole receptor serotonin agonist and thereby make the formulation organoleptically acceptable.

As summarized above, the subject formulations are orally acceptable formulations. The formulations may be present in a number of different formats, where representative formats include, but are not limited to sublingual formulations, such as: lozenges, tablets, semi-solid formulations such as oral films, gels, and gums. In certain embodiments, the composition is one that is configured to be dispensed to the buccal or sublingual surfaces. Formulations suitable for buccal/sublingual delivery include in a number of different formulations or dosage forms including, but not limited to, fast-melting tablets, liquid-filled capsules, liquid sprays or lozenges. Alternatively, a pharmaceutical composition can be delivered to the mucosa of the oral cavity by direct placement of the composition in the mouth, for example, with a gel, a film, an ointment, a dropper, or a bioadhesive strip or patch. The term “lozenge” as used herein is intended to embrace all dosage forms (including troches) where the product is formed by cooling a sugar-based or sugar alcohol based (e.g., sorbitol) molten mass containing the active material. The term “tablet” as used herein is intended to embrace unit dosage forms made from compressed powders or granules or compressed pastes.

In certain embodiments, the compositions may include a flavoring agent. Flavoring agents that may be used in the present invention include, and are not limited to, natural flavors, natural fruit flavors, artificial flavors, artificial fruit flavors, flavor enhancers or mixtures thereof. Natural flavors, artificial flavors or mixtures thereof include, and are not limited to, mint (e.g., peppermint or spearmint), lemon, lime, orange, strawberry, menthol, cinnamon, vanilla, artificial vanilla, chocolate, artificial chocolate or bubblegum. Natural fruit flavors, artificial fruit flavors or mixtures thereof include, and are not limited to, cherry, grape, orange, strawberry or lemon. Flavor enhancers include, and are not limited to, citric acid. Although flavoring agents are generally provided as a minor component of the taste masking composition in amounts effective to provide a palatable flavor to the liquid pharmaceutical composition, the addition of at least one flavoring agent is preferred; and, more preferably, up to two flavoring agents may be employed. A flavoring agent used in the taste masking composition has a range of from about 0.01 to about 0.15 grams per 100 mL. The flavorings are generally utilized in amounts that will vary depending upon the individual flavor, and may, for example, range in amounts of about 0.01% to about 10% by weight/volume of the final composition.

Examples of sweeteners include sweetening agents, artificial sweeteners and dipeptide based sweeteners, e.g., monosaccharides, disaccharides and polysaccharides such as xylose, ribose, glucose, mannose, galactose, fructose, dextrose, sucrose, sugar, maltose, partially hydrolyzed starch, or corn syrup solids and sugar alcohols such as sorbitol, xylitol, mannitol, saccharin salts, i.e., sodium, or calcium saccharin salts, cyclamate salts, acesulfam-K, ammonium glycyrrhizinate, dipotassium glycyrrhizinate and the free acid form of saccharin L-aspartylphenylalanine methyl ester and mixtures thereof.

When present, the sweetener may be present in an amount corresponding to about 1 to 60% weight/volume of the total composition, the amount depending in part upon whether other sweetener ingredients are present and the level of sweetness desired. Typically sugar is used it is present from about 10% to about 50% w/v of the composition. It will be appreciated that combinations of sweeteners can be used. The sweetening agents, when used, may also be used alone or in combination with each other. When an artificial sweetness enhancer is used it may be present in an amount from about 0.05% to about 15% weight/volume of the final composition.

Certain embodiments of the formulations may include a colorant. Colorants useful in the present invention include pigments such as titanium dioxide, that may be incorporated in amounts of up to about 10% by weight/volume. The colorants may include other dyes suitable for food, drug and cosmetic applications, and known as F.D. & C. dyes and the like. The materials acceptable for the foregoing spectrum of use may be water-soluble. Illustrative examples include indigoid dye, known as F.D. & C. Blue No. 2, which is the disodium salt of 5,5′-indigotindisulfonic acid. Similarly, the dye known as F.D. & C. Green No. 1, includes a triphenylmethane dye and is the monosodium salt of 4-[4-Nethyl-p-sulfobenzylamino)diphenylmethylene]-[1-(N-ethyl-N-p-sulfoni- umbenzyl)-2,5-cyclohexadienimine].

Any convenient fabrication protocol may be employed to prepare the subject formulations. Solid dosage forms may be prepared by methods which are well known in the art for the production of lozenges, tablets, troches, capsules or chewing gums and may contain other ingredients known in such dosage forms such as acidity regulators, opacifiers, stabilizing agents, buffering agents, flavorings, sweeteners, coloring agents, buffering agents, sweeteners and preservatives.

For example, solid formulations of the present invention may be prepared as lozenges by heating the lozenge base (e.g., a mixture of sugar and liquid glucose) under a vacuum to remove excess water. The remaining components are then blended into the mixture. The resulting mixture is then drawn into a continuous cylindrical mass from which the individual lozenges are formed. The lozenges are then cooled, subjected to a visual check and packed into suitable packaging. One form of suitable packaging is a blister pack of a water-impermeable plastics material (e.g., polyvinylchloride) closed by a metallic e.g., aluminium foil. The patient removes the lozenge by applying pressure to the blister to force the lozenge to rupture and pass through the metal foil seal. Where desired, ethanol can be used to dissolve components of the formulation.

Masticable solid dosage formulations may be made by the methods used to prepare chewable candy products or chewing gums. For example, a chewable solid dosage form may be prepared from an extruded mixture of sugar syrup to which the ibuprofen has been added with optional addition of whipping agents, humectants, lubricants, flavors and colorings. (See Pharmaceutical Dosage Forms: Tablets, Volume 1, Second Edition edited by H A Lieberman, L Lachman and J B Schwartz published in 1989).

As such, a variety of different oral dosage formulations are provided by the subject invention. Furthermore, the oral dosage formulations do not need a special procedure for their preparation, as they may be readily produced using conventional procedures. For example, taste-masking agents, diluents, binders, or other appropriate additives can be added to indole serotonin receptor agonist, to which water or organic solvents are added, if necessary, and then mixed evenly to be compacted or to be granulated, and then mixed with lubricant to be compacted. For a diluent, sugar is mainly used and one or more types of sugar such as white sugar, powder sugar, lactose, fructose, starch syrup, reduced malt sugar, D- mannitol, D-sorbitol, and sucrose. For a binder, polyvinyl pyrrolidone, hydroxypropylcellulose, hydroxypropylmethylcellulose, corn starch, gelatin and arabic gum are used. For a lubricant, magnesium stearate, talc, sucrose fatty acid ester and such are properly selected and used.

In certain embodiments, the methods of manufacture may be characterized by including a first step of producing an intermediate composition, which composition includes the active agent and masking component, and then a second step of producing the oral dosage formulation from the intermediate composition.

### Methods of Use

The present invention provides methods of delivering a therapeutic amount of an indole serotonin receptor agonist to an individual in need thereof. Aspects of the methods include administering an oral dosage formulation to an individual. In practicing the invention, the dosage may be placed in the mouth of the subject, e.g., by the subject itself or a caregiver therefore, whereupon the subject holds the formulation in its mouth to obtain the desired benefit, where the term holding is used broadly to include sucking, chewing, maintaining, etc, depending on the particular type of formulation, so that the active agent is systemically administered to the patient.

In practicing the subject methods, a formulation may be administered a single time or a plurality of times over a given time period, e.g., the course of the disease condition, being treated, where the dosing schedule when a plurality of formulations are administered over a given time period may be hourly, daily etc. Aspects of the invention include delivery the composition to an individual via a buccal or sublingual route.

In some aspects of the present invention, the methods comprise administering to an individual a pharmaceutical composition wherein administration, to the buccal and/or sublingual mucosal surfaces of the oral cavity is by a delivery device. The delivery device can include, but is not limited to, unit dose containers, pump sprays, droppers, squeeze bottles, airless and preservative-free sprays, nebulizers, dose inhalers and pressurized dose inhalers. The delivery device can be metered to administer an accurate effective dosage amount (as described below) to the oral cavity. In some aspects, an accurate effective dosage amount is contained within a capsule, tablet, lozenge, or bioadhesive patch that is placed directly within the oral cavity.

Dosages can be administered in a single dose or in multiple doses, for example, dosages can be administered two, three, four, up to ten times daily depending on the type and severity of headache pain being treated as well as on individual susceptibility. Dosages can be administered in a sustained release formulation which may allow for an oxytocin peptide to be administered less frequently such as six times a week, five times a week, four times a week, three times a week, twice a week, or once a week.

A subject delivery method will, in certain embodiments, provide a therapeutic level of an indole serotonin receptor agonist, e.g., a level of an indole serotonin receptor agonist that is sufficient to inhibit, prevent, or reduce headache pain. By “therapeutic level” is meant a level in plasma or other internal bodily tissue or fluid (e.g., cranial fluid, cerebrospinal fluid) that provides for reduction, inhibition, or prevention of headache pain.

Generally, subjects to which the subject formulations may be administered are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In certain embodiments, the subjects will be humans.

In some embodiments, a subject delivery method treats a headache, e.g., the method is suitable for abortive therapy of a headache. In other embodiments, a subject delivery method prevents the occurrence of a headache. In some embodiments, a subject delivery method reduces or eliminates one or more symptoms of a migraine headache.

Individuals who are suitable for treatment with a subject delivery method include individuals suffering from migraine headache; and individuals who are prone to suffering from migraine headaches, e.g., individuals with a history of migraine headache. Individuals who are suitable for treatment with a subject delivery method also include individuals suffering from a rebound headache. Individuals who are suitable for treatment with a subject delivery method also include individuals suffering from status migrainosus. Individuals may be diagnosed as being in need of the subject methods using any convenient protocol, and are generally known to be in need of the subject methods prior to practicing the subject methods. In certain embodiments, the methods include a step of diagnosing the presence of a headache and then administering a formulation of the invention to treat the headache, e.g., where treat means at least diminishing the pain of the headache to some extent, if not eliminate the pain of the headache.

### Kits

Also provided are kits, where the subject kits at least include one or more, e.g., a plurality of, organoleptically acceptable oral dosage formulations, as described above. The subject formulations in the kits may be present in a package. The formulations of the kits may be present in individual pouches or analogous containers, to preserve the composition of the formulations until use.

The subject kits may also include instructions for how to use the formulations, where the instructions typically include information about how to administer the formulation, dosing schedules etc. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g. CD-ROM, diskette, etc.

The following practical and comparative examples are offered by way of illustration and not by way of limitation.

### EXAMPLES

### I. Sample and Preparation Method

An appropriate amount of zolmitriptan, sumatriptan succinate, and bitterness-masking compound is measured to realize each concentration showed in Table 1. Each measured amount is placed in a 50 mL measuring flask and 10 mL KC1 is added to the flask to make 50 mL to prepare each sample solution.

TABLE 1
Evaluation Sample Composition

| | | Drug Conc. | Masking | Comp. Conc. |
|---|---|---|---|---|
| No. | Drug Name | [ppm] | Compound | [ppm] |
| 1 | Zolmitriptan | 100 | — | — |
| 2 | Zolmitriptan | 100 | glutamic acid | 200 |
| 3 | Zolmitriptan | 100 | malic acid | 300 |
| 4 | Zolmitriptan | 100 | citric acid | 300 |
| 5 | Zolmitriptan | 100 | ascorbic acid | 300 |
| 6 | Sumatriptan | 100 | — | — |
| 7 | Sumatriptan | 100 | glutamic acid | 200 |

30 mM KCl+0.3 mM tartaric acid solution was prepared as a reference solution.

### II. Measurement by Taste Sensor and Data Analysis

A. The principle of the Taste Sensor

A taste sensor, SA402B (Intelligent Sensor Technology Inc., Japan)(See e.g., Myanaga et a., Sensors and Materials (2002) 8:455-465; and Nakamura et al., Chem. Pharm. Bull (2002) 50:1589-1593), was used in this test. This device includes an electrode part that has a lipid membrane sensor, a robot arm, and a computer. The electrode part consists of the lipid membrane sensor and the reference electrode. The potential difference between each sensor and the reference electrode becomes an output and this signal is sent to the computer through the robot arm. The lipid membrane sensor can be selected according to the drug to measure and six sensors were used in this test. This devise mimics the human gustatory mechanism where various types of sensation can be felt through the various receptors existing in the taste cells of the tongue. It is possible to obtain a sensor response pattern to different types of bitterness (Acidic bitterness, Aftertaste from acidic bitterness, Basic bitterness (1), Basic bitterness (2), Aftertaste from astringency, Astringency) by preparing many types of lipid membrane sensors with different membrane compositions. When the lipid membrane sensor part is dipped into the sample solution of the bitter drug, the lipid membrane potential changes due to the static mutual mechanism between the drug molecules and the lipid membrane as well as the physical adsorption of the drug into the lipid membrane while the signal is retrieved as the information. This is the principle of the measurement.

The lipid membrane used in this test is a combination of polyvinyl chloride, a plasticizer and lipid. The components of the lipid membrane in each sensor are showed in Table 2.

TABLE 2
Sensor Lipid Membrane Composition and Corresponding Tastes

| Sensor # | Lipid Membrane Composition | Taste |
|---|---|---|
| 1 | Hexadecanoic acid, Dioctyl phenyl- | Basic bitterness 1 |
| | phosphonate | |
| 2 | Phosphoric acid di-n-decyl ester, Dioctyl | Basic bitterness 2 |
| | phenyl-phosphonate | |
| 3 | Tetradodecyl ammonium bromide, Dioctyl | Aftertaste from |
| | phenyl-phosphonate | astringency |
| 4 | Tetradodecyl ammonium bromide, Dioctyl | Astringency |
| | phenyl-phosphonate | |
| 5 | Tetradodecyl ammonium bromide, | Acidic bitterness |
| | 2-Nitrophenyl octyl eter | |
| 6 | Tetradodecyl ammonium bromide, | Aftertaste from |
| | 2-Nitrophenyl octyl eter | acidic bitterness |

To produce a specific output pattern, the lipid in the first and the second sensors has a negative charge because of the phosphate group while the lipid in the third through the sixth sensors has a positive charge because of the ammonium group

### B. Measurement Method

The membrane potential of each sample solution was measured in the following procedure.

The membrane potential Vr (mV) of the reference solution was measured before measuring the sample solution. 30 mM KCl+0.3 mM tartaric acid solution, which is equivalent to the human saliva, almost tasteless and makes the output of the taste sensor stable, was used as the reference solution. Next, the membrane potential of the sample solution Vs (mV) was measured. After measuring the membrane potential of the sample solution and washing the sensor away with the reference solution, the membrane potential of the reference solution Vr′ (mV) was measured again. After this measurement, the sensor was thoroughly cleaned with 30% ethanol solution to make it the initial condition.

As the reference solution is equivalent to the human saliva, the potential difference from the sample solution of the bitter drug (Vs−Vr) is the value to evaluate the taste. The change of the membrane potential (Vr′−Vr) before and after measuring the sample solution seems to be caused by the adhesion of the bitter drug to the lipid membrane. This change is a CAP (Change of membrane Potential caused by Adsorption) value, representing bitterness and astringency that remains for a while after orally taking bitter drugs.

### C. Data Analysis

Weber's principle teaches that human beings can distinguish the strength of taste when the difference in concentration between two given taste samples is 20%. In other words, we can recognize a difference in tastes when the difference of concentration is 1.2 times. Therefore, the difference of taste with a 10-time concentration is equivalent to 1.2^{12.6} times.

The membrane potential Vs (mV) was measured on the assumption that the membrane potential of each solution is the same as that of the reference solution, since the experiences until the present show that the basic bitterness (1) and (2) of 0.01 mM quinine hydrochloride solution, the astringency of 0.001% iso-alpha-acid solution, and the acidic bitterness of 0.0005 tannic acid solution have no taste. The membrane potential Vs (mV) was measured for the solution with a 10-time concentration for each solution (0.1 mM quinine hydrochloride solution, 0.01% iso-alpha-acid solution, 0.005% tannic acid solution) as well. The potential difference between the reference solution and each solution with a 10-time concentration was obtained and it was divided by 12.6, following Weber's principle, to be 1 division of the taste scale.

Based on this number, the numerical taste value was calculated from the potential difference between the sample solution and the reference solution of the bitter drug. The results are showed in Table 3.

As seen in the working examples and comparison examples above, the bitterness of Zolmitriptan consists of acidic bitterness, astringency, basic bitterness 1 and basic bitterness 2. It was confirmed that each element in the bitterness of Zolmitriptan is reduced by adding glutamic acid, malic acid, citric acid and ascorbic acid (as evidenced by the declining number in Table 3).

In a similar way, the bitterness of Sumatriptan consists of astringency, basic bitterness 1 and basic bitterness 2. It was confirmed that each element in the bitterness of Sumatriptan is reduced by adding glutamic acid (as evidenced by the declining number in Table 3).

TABLE 3
Bitterness
Measurement Data

| | | | | | Aftertaste from | |
|---|---|---|---|---|---|---|
| | Acidic | | Basic | Basic | Acidic | Aftertaste |
| | Bitterness | Astringency | bitterness 1 | bitterness 2 | Bitterness | from Astringency |
| Zolmitriptan | 8.12 | 4.68 | 2.67 | 1.44 | 0.12 | −0.07 |
| Zolmi + | −2.61 | 1.86 | 0.39 | −1.36 | −1.36 | −0.03 |
| glutamic acid | | | | | | |
| Zolmi + malic | −2.53 | 2.94 | 0.23 | −1.85 | −1.42 | −0.05 |
| acid | | | | | | |
| Zolmi + citric | −1.49 | 1.45 | 0.23 | −0.37 | 0.38 | 0.14 |
| acid | | | | | | |
| Zolmi + | −0.82 | 0.57 | −0.13 | −0.46 | 0.32 | 0 |
| ascorbic acid | | | | | | |
| Sumatriptan | −2.96 | 0.78 | 0.35 | 2.45 | −0.84 | −0.06 |
| Suma + | −4.71 | −0.14 | −0.22 | 0.54 | −0.88 | −0.28 |
| glutamic acid | | | | | | |

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. An organoleptically acceptable oral dosage formulation of an indole serotonin receptor agonist, said formulation comprising:
an indole serotonin receptor agonist; and
a masking component, wherein said masking component is an amino acid or an organic acid.

2. The formulation according to claim 1, wherein said masking component is an amino acid.

3. The formulation according to claim 2, wherein said amino acid is glutamic acid or glycine.

4. The formulation according to claim 1, wherein said masking component includes an organic acid.

5. The formulation according to claim 4, wherein said organic acid is chosen from citric acid, malic acid and ascorbic acid.

6. The formulation of claim 1, wherein said indole serotonin receptor agonist is selected from sumatriptan, frovatriptan, zolmitriptan, eletriptan, rizatriptan, naratriptan, and almotriptan or a pharmaceutically acceptable salt thereof.

7. The formulation according to claim 1, wherein said oral dosage formulation is an oral film, lozenge, tablet, gel or gum.

8. A method of delivering a therapeutic amount of an indole serotonin receptor agonist to an individual in need thereof, the method comprising:
administering to said individual an organoleptically acceptable oral dosage formulation of an indole serotonin receptor agonist, said formulation comprising:
an indole serotonin receptor agonist; and
a masking component, wherein said masking component is an amino acid or an organic acid.

9. The method according to claim 8, wherein said administering comprises introducing said formulation sublingually to said individual.

10. The method according to claim 8, wherein said masking component is an amino acid.

11. The method according to claim 10, wherein said amino acid is glutamic acid or glycine.

12. The method according to claim 8, wherein said masking component includes an organic acid.

13. The method according to claim 12, wherein said organic acid is chosen from citric acid, malic acid and ascorbic acid.

14. The method according to claim 8, wherein said indole serotonin receptor agonist is selected from sumatriptan, frovatriptan, zolmitriptan, eletriptan, rizatriptan, naratriptan, and almotriptan or a pharmaceutically acceptable salt thereof.

15. The method according to claim 8, wherein said oral dosage formulation is an oral film, lozenge, tablet, gel or gum.

16. The method according to claim 8, wherein said method is a method of treating a headache.

17. The method according to claim 8, wherein said method is a method of preventing a headache.

18. The method of claim 8, wherein the method provides for a level of indole serotonin receptor agonist in the individual that is effective to inhibit migraine pain.

19. A kit comprising an organoleptically acceptable oral dosage formulation of an indole serotonin receptor agonist.

20. The kit according to claim 19, wherein said oral dosage formulation is a lozenge, tablet, gel or gum.
